(19) United States
(12) Patent Application Publication
(10) Pub. No.: US 2006/0110438 A1
(43) Pub. Date: May 25, 2006

(54) Liposome stabilizing agent

(76) Inventors: Makoto Suematsu, Tokyo (JP); Kazuhiro Suganuma, Tokyo (JP)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/996,740

(22) Filed: Nov. 24, 2004

(51) Int. Cl. A61K 9/127; C07C 235/12

(52) U.S. Cl. 424/450; 554/35

(57) ABSTRACT

The present invention provides a liposome stabilizing agent comprising a lipid of carboxylic acid type represented by the following general formula: 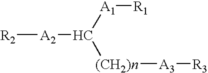 (wherein
any one of R1, R2 and R3 represents the following general formula: 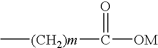 [wherein M represents a hydrogen atom or a monovalent cation and m, which defines the methylene chain length, represents an integer of 1 to 5], and the other two each represent an chain hydrocarbon group, A1, A2 and A3 are each independently selected from the group consisting of C(O)O, CONH and NHCO, and n, which defines the methylene chain length, represents an integer of 1 to 3), as well as liposomes containing the liposome stabilizing agent and a kit comprising the liposome stabilizing agent.

### FIELD OF THE INVENTION

The present invention relates to a liposome stabilizing agent comprising a lipid of carboxylic acid type.

### BACKGROUND OF THE INVENTION

Liposomes are vesicles that are artificially formed in analogy to biomembranes such as cell membranes. Liposomes are used as carriers for gene transfer in gene therapy, oxygen carriers encapsulating erythrocytes therein, drug carriers containing water-soluble and/or fat-soluble drugs, etc.

Their typical structure is a lipid bilayer. For example, a mixed suspension composed of membrane-constituting lipids (e.g., cholesterol) and phospholipids (e.g., dipalmitoylphosphatidylglycerol (DPPG), dipalmitoylphosphatidylcholine (DPPC)) may be vigorously stirred to give vesicles made of lipid bilayers. In this case, phospholipids serve as membrane stabilizers, and hence in the absence of phospholipids, the membrane structure will become unstable because trilayers or other structures are formed.

### SUMMARY OF THE INVENTION

However, since phospholipids have a platelet-activating effect and a leukocyte adhesion-activating effect in vivo, the administration of phospholipid-containing liposomes will produce side effects such as thrombogenesis, thrombocytopenia and leukocyte dysfunction. This may be a big barrier to using liposome formulations, particularly as oxygen carriers in hemorrhagic shock conditions and/or as carriers of drugs (e.g., nucleic acids, carcinostatic agents) for gene transfer in cancer patients with low platelet counts.

For this reason, the object of the present invention is to provide a liposome stabilizing agent without any disturbance effects on microcirculation, such as a platelet-activating effect and a leukocyte adhesion-activating effect, as well as to provide liposomes containing the liposome stabilizing agent and a kit comprising the liposome stabilizing agent.

Recently, lipids of carboxylic acid type free from phosphoric acid groups have been developed as amphipathic lipids. The inventors of the present invention have focused on the fact that these lipids are free from phosphoric acid groups, and have used them as liposome stabilizing agents for the first time, thus finding that these lipids do not cause any disturbance effects on microcirculation in vivo, such as a platelet-activating effect and a leukocyte adhesion-activating effect. This finding led to the completion of the present invention.

The liposome stabilizing agent of the present invention comprises a lipid of carboxylic acid type represented by the following general formula: 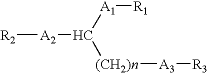
(wherein

any one of R1, R2 and R3 represents the following general formula: 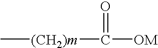
[wherein M represents a hydrogen atom or a monovalent cation and m, which defines the methylene chain length, represents an integer of 1 to 5], and the other two each represent an chain hydrocarbon group,

A1, A2 and A3 are each independently selected from the group consisting of C(O)O, CONH and NHCO, and

n, which defines the methylene chain length, represents an integer of 1 to 3).

The liposome stabilizing agent of the present invention may also be characterized in that liposomes containing the same will have no platelet-activating effect in vivo. As used herein, the term “platelet-activating effect” refers to the ability to activate platelets, and may be an effect that specifically causes some reaction, e.g., activation of signal transduction systems through platelet membrane glycoprotein Ib (GPIb), upon platelet activation.

The liposome stabilizing agent of the present invention may also be characterized in that in vivo administration of liposomes containing the same will cause no reduction of platelet counts in the circulating blood.

The liposome stabilizing agent of the present invention may also be characterized in that liposomes containing the same will cause no transient adhesion of platelets in vivo.

The liposome stabilizing agent of the present invention may also be characterized in that liposomes containing the same will have no leukocyte adhesion-activating effect in vivo.

As used herein, the above recognition meaning that liposomes do not have certain effects or functions, such as having no platelet-activating effect, causing no reduction of platelet counts in the circulating blood, causing no transient adhesion of platelets and having no leukocyte adhesion-activating effect, are defined as showing no statistically significant difference over control, as determined by statistical evaluation using the chi-square test, the t-test, etc.

The liposome stabilizing agent of the present invention may also be characterized in that the lipid of carboxylic acid type contained therein is DPEA. The chemical formula of DPEA is shown below.

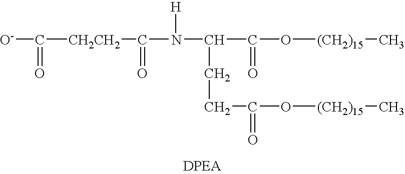

Further, the liposomes of the present invention contain any one of the liposome stabilizing agents defined above. The liposomes of the present invention may further comprise a compound for disease treatment.

Furthermore, the kit of the present invention comprises any one of the liposome stabilizing agents defined above.

### BRIEF DESCRIPTION OF THE DRAWINGS

FIG. 1 is a graph showing the results of transient platelet adhesive activity measured in one embodiment of the present invention. The line segment perpendicular to each data plot represents standard deviation. The asterisk (*) indicates statistically significant different data.

FIG. 2 is a graph showing the results of leukocyte adhesive activity measured in one embodiment of the present invention. The line segment perpendicular to each data plot represents standard deviation. The asterisk (*) indicates statistically significant different data.

FIG. 3 is a graph showing the results of leukocyte adhesive activity measured in one embodiment of the present invention. The line segment perpendicular to each data plot represents standard deviation. The asterisk (*) indicates statistically significant different data.

### DETAILED DESCRIPTION OF THE INVENTION

The present invention provides a liposome stabilizing agent comprising a lipid of carboxylic acid type represented by the following general formula: 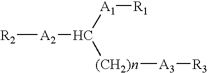
(wherein

any one of R1, R2 and R3 represents the following general formula: 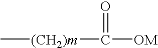
[wherein M represents a hydrogen atom or a monovalent cation and m, which defines the methylene chain length, represents an integer of 1 to 5], and the other two each represent an chain hydrocarbon group,

A1, A2 and A3 are each independently selected from the group consisting of C(O)O, CONH and NHCO, and

n, which defines the methylene chain length, represents an integer of 1 to 3). Binding sites for R1, R2 and R3 are preferably trifunctional amino acids such as lysine, asparagine, glutamine, aspartic acid, glutamic acid, serine, threonine and tyrosine.

In particular, preferred are trifunctional amino acids having one reactive functional group and two other identical reactive functional groups, i.e., those having one terminal amino group and two terminal carboxyl groups, as exemplified by aspartic acid and glutamic acid, or those having one terminal carboxyl group and two terminal amino groups, as exemplified by lysine, asparagine and glutamine. Particularly preferred are aspartic acid and glutamic acid. Likewise, homocysteine, glutathione and the like may also be used for this purpose. In the EXAMPLES section described later, a compound whose binding sites for R1, R2 and R3 are each glutamic acid is used.

Preparation of liposomes containing the above lipid of carboxylic acid type may be accomplished in a standard manner. For example, the above lipid of carboxylic acid type may be used as a liposome stabilizing agent and mixed with cholesterol, and the resulting mixed aqueous suspension is treated by ultrasonication or vigorously shaken or stirred to prepare a liposome solution containing lipid bilayer liposomes with a diameter of 0.1 to 1 μm. At this time, PEG (polyethylene glycol)-modified cholesterol or the like may further be incorporated as a surface modifier to achieve PEG modification on the surface of liposomes. This allows an increase in the blood retention properties of liposomes.

In a case where a compound for disease treatment or the like is intended to be contained in liposomes, such a compound may be added in advance during liposome solution preparation to allow the compound to be incorporated into liposomes. Examples of compounds for disease treatment include nucleic acids, amino acids, proteins, antitumor agents, anti-inflammatory agents, anti-infective agents, biological, immunological or hematological agents, dermatologic agents, agents for sensory organs, agents for the endocrine system, gastrointestinal agents, cardiovascular agents, agents for excretory organs, genital agents, respiratory agents, agents for the nervous system, diagnostic aids, and nutrient preparations. Specific examples include a wide variety of pharmaceutical preparations such as DNA and RNA for use in gene therapy, hemoglobin for use in artificial erythrocytes, and cancer chemotherapeutics.

The liposomes thus prepared may then be administered to vertebrate animals including mammals such as human, mouse and rat. Administration may be accomplished in various forms depending on the intended purpose, including intravenous administration, intraperitoneal administration, intramuscular administration, subcutaneous administration, transnasal administration, transpulmonary administration, oral administration, and external application. Alternatively, the liposomes may also be administered in combination with a pharmaceutically acceptable excipient or base, depending on the form of administration.

In the EXAMPLES section described later, rats were intravenously administered.

Next, the animals administered with the liposomes are examined for disturbance effects on microcirculation caused by the administered liposomes. Administration of phospholipid-containing liposomes will trigger platelet activation. During platelet activation, platelet membrane glycoprotein GPIIb/IIIa is first activated by intracellular signals generated upon binding between platelet membrane glycoprotein Ib (GPIb) and adhesive protein vWF (von Willebrand factor). When bound to the adhesive protein, the activated GPIIb/IIIa further causes platelet-platelet binding, thus resulting in platelet adhesion and aggregation. In addition, P-selectin expression is induced on the activated platelet cell membranes. The expressed P-selectin adheres to sLex, a sugar chain on leukocyte membranes, and contributes to leukocyte rolling together with P-selectin expressed on vascular endothelial cell membranes.

In this process, transient platelet adhesion and leukocyte adhesion are observed as disturbance effects on microcirculation. Transient platelet adhesion may be detected, e.g., by observing platelets stained with a fluorescent material or by determining platelet counts in the circulating blood, both of which may be used as indicative of transient platelet adhesion. In the EXAMPLES section, carboxyfluorescein diacetate succinimidyl ester (CFSE, a product of Molecular Probes, Inc.) was used for platelet staining. This material is converted into a fluorescent dye by the action of esterase in platelets to ensure fluorescent staining of platelets. On the other hand, leukocyte adhesion may be detected, e.g., by measuring the energy of platelet-mediated adhesion between leukocytes and vascular endothelial cells or by determining the counts of leukocytes adhered to the microvascular wall, both of which may be used as indicative of leukocyte adhesion. In the EXAMPLES section, the counts of leukocytes adhered to the microvascular wall were determined as indicative of leukocyte adhesion.

As shown in the EXAMPLES section below, when liposomes containing DPEA (one of the lipids of carboxylic acid type) are administered to rats, their disturbance effects on microcirculation are not observed. This indicates that DPEA is a liposome stabilizing agent having no disturbance effect on microcirculation. If this liposome stabilizing agent is packaged into a kit together with, for example, a necessary reagent such as cholesterol, such a kit allows experimenters or physicians to use the present invention conveniently.

### EXAMPLES

One embodiment of the present invention will be further described in more detail below.

<Preparation of Liposomes>

Liposomes containing DPPG as a stabilizing agent were prepared in a standard manner (see J. Biochem. vol. 131, p. 611-617, 2002; Bioconjugate Chem. vol. 8, p. 23-30, 1997) under the conditions shown in Table 1. In this Example, the same mol % of PA or DPEA was also added instead of DPPG to prepared liposomes.

TABLE 1

| | PC/CH/DPPG | PC/CH/PA | PC/CH/DPEA |
|---|---|---|---|
| Ratio | 5:5:1 | 5:5:1 | 5:5:1 |
| Lipid (wt %) | 5 | 5 | 5 |
| Number of animals | 6 | 3 | 7 |
| pH | 7.4 | 7.4 | 7.4 |
| PEG | 0.3 mol % | 0.3 mol % | 0.3 mol % |

Abbreviations:
PC, phosphatidylcholine;
CH, cholesterol

The chemical formulae of DPPG and PA are shown below. 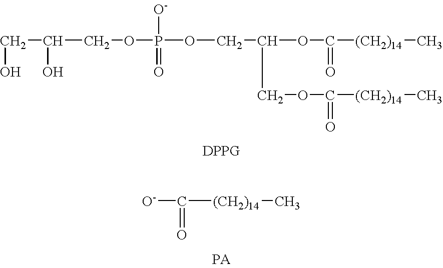
<In Vivo Labeling of Rat Platelets>

Male Wistar rats (body weight: 280 to 300 g) were anesthetized by intramuscular injection of pentobarbital sodium (trade name: Nembutal) into their femoral muscle in an amount of 50 mg per kg body weight. The femoral vein of each rat was exposed and incised to insert a 3Fr Atom-tube, followed by suturing of the incision site on the skin. This Atom-tube was injected with 200 μl of 15 mM CFSE to effect in vivo fluorescent labeling of platelets. Next, the intestine and mesentery were exposed extraperitoneally by abdominal midline incision. The mesentery was treated by surface perfusion using Krebs-Henseleit buffer (37° C., pH 7.4) saturated with 95% N₂-5% CO₂ mixed gas.

<Administration and Observation of Liposomes>

The mesentery of each rat was mounted on the stage of an upright video biomicroscope and allowed to stand for 10 minutes to stabilize the sample, followed by administration of a DPEA-containing liposome solution (2 ml) from upstream at a flow rate of 0.4 ml/minute for 5 minutes. As controls, a vehicle alone (liposome-free), a palmitic acid (PA)-containing liposome solution and a DPPG-containing liposome solution were used to perform the same experiment.

At 5, 10, 20 and 40 minutes after initiation of administration, the mesenteric microcirculation (venule) was observed on a video monitor with transmitted light and fluorescence (wavelength 488 nm). When observing with an objective lens of 40 magnifications, CFSE-labeled platelets will emit fluorescent signals of needle tip size, while CFSE-labeled leukocytes will be distinguished as fluorescence-emitting larger spherical cells.

<Evaluation of Transient Platelet Adhesive Activity>

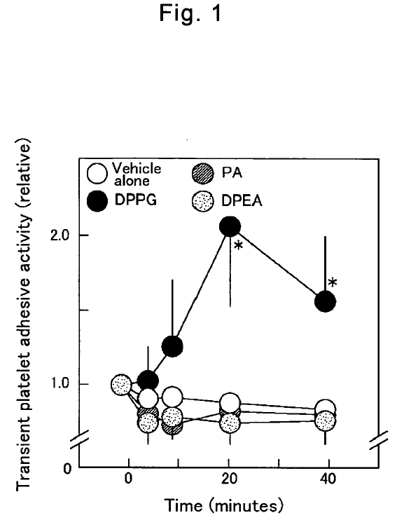

Once platelets are activated, platelet-platelet adhesion will be observed transiently. Under the experimental conditions described above, to quantify transient platelet adhesive activity, adhered cells were counted immediately before and 5, 10, 20 and 40 minutes after initiation of liposome solution administration. Assuming that the adhered cell counts immediately before initiation of administration was set to 1, adhered cell counts at each time point were expressed as relative ratio and plotted on a graph. FIG. 1 shows the results obtained.

When the DPPG-containing liposome solution was administered, transient platelet adhesion occurred and reached a maximum activity level (about twice as high as before administration) at 20 minutes after liposome administration. However, when the vehicle alone, the palmitic acid (PA)-containing liposome solution or the DPEA-containing liposome solution was administered, no transient platelet adhesion was observed.

<Evaluation of Leukocyte Adhesive Activity>

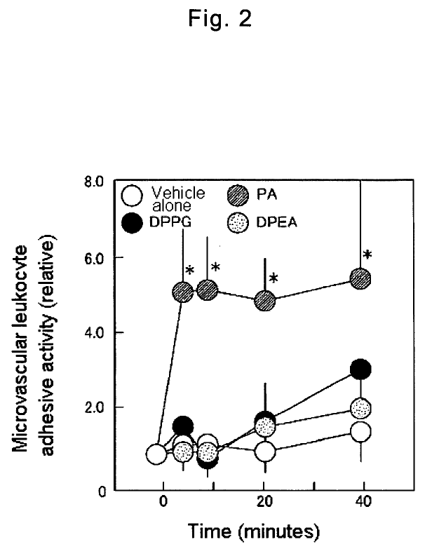

In response to platelet activation, leukocytes will adhere to vascular endothelial cells in the vascular wall. Under the experimental conditions described above, to evaluate leukocyte adhesive activity, cells adhered to the vascular wall were counted immediately before and 5, 10, 20 and 40 minutes after initiation of liposome solution administration. Assuming that the adhered cell counts immediately before initiation of administration was set to 1, adhered cell counts at each time point were expressed as relative ratio and plotted on a graph. FIG. 2 shows the results obtained.

When the PA-containing liposome solution was administered, leukocyte adhesion to the vascular wall occurred and reached a maximum activity level (about 5 times as high as before administration) at 5 minutes after liposome administration, with no change after 40 minutes.

However, when the vehicle alone, the DPPG-containing liposome solution or the DPEA-containing liposome solution was administered, no leukocyte adhesive activity was observed.

<Measurement of Blood Platelet Concentration>

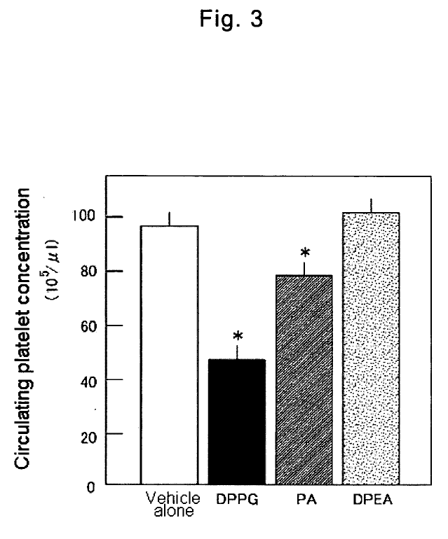

After completion of these experiments, 1 ml blood was collected from the large vein and platelet counts per unit volume (platelet concentration) were measured for each experimental condition. FIG. 3 shows the results obtained.

When compared to administration of the vehicle alone, administration of the DPPG- or PA-containing liposome solution resulted in a reduction of blood platelet concentration, whereas administration of the DPEA-containing liposome solution caused no change in platelet concentration.

### INDUSTRIAL APPLICABILITY

The present invention enables the provision of a liposome stabilizing agent without any disturbance effects on microcirculation, such as a platelet-activating effect and a leukocyte adhesion-activating effect, as well as liposomes containing the liposome stabilizing agent and a kit comprising the liposome stabilizing agent.

1. A liposome stabilizing agent comprising a lipid of carboxylic acid type represented by the following general formula: 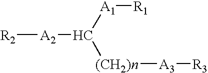
wherein any one of R1, R2 and R3 represents the following general formula: 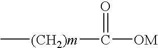
wherein M represents a hydrogen atom or a monovalent cation and m, which defines the methylene chain length, represents an integer of 1 to and the other two each represent a chain hydrocarbon group,
A1, A2 and A3 are each independently selected from the group consisting of C(O)O, CONH and NHCO, and
n, which defines the methylene chain length, represents an integer of 1 to 3.

2. The liposome stabilizing agent according to claim 1, wherein a liposome containing the liposome stabilizing agent will have no platelet-activating effect in vivo.

3. The liposome stabilizing agent according to claim 1 or 2, wherein in vivo administration of a liposome containing the liposome stabilizing agent will cause no reduction of platelet counts in the circulating blood.

4. The liposome stabilizing agent according to claim 1 or 2, wherein a liposome containing the liposome stabilizing agent will cause no transient adhesion of platelets in vivo.

5. The liposome stabilizing agent according to claim 1, wherein a liposome containing the liposome stabilizing agent will have no leukocyte adhesion-activating effect in vivo.

6. The liposome stabilizing agent according to claim 1 wherein the lipid of carboxylic acid type is DPEA (1,5-dipalmitoyl-L-glutamate-N-succinic acid).

7. A liposome containing the liposome stabilizing agent according to claim 1.

8. The liposome according to claim 7, which comprises a compound for disease treatment.

9. A kit comprising the liposome stabilizing agent according to claim 1.
